(19) United States
(12) Patent Application Publication
(10) Pub. No.: US 2008/0071144 A1
(43) Pub. Date: Mar. 20, 2008

(54) NOVEL ENHANCED HIGHER DEFINITION ENDOSCOPE

(76) Inventor: William Fein, Beverly Hills, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/855,969

(22) Filed: Sep. 14, 2007

Related U.S. Application Data
(60) Provisional application No. 60/825,893, filed on Sep. 15, 2006.

(51) Int. Cl. A61B 1/06

(52) U.S. Cl. 600/178

(57) ABSTRACT

Novel enhanced high definition endoscopy is facilitated by multiple illumination sources and a plurality of illumination sources and a plurality of illumination zones, whereby improved images are generated and image date output is communicated to eye pieces, computer screens, cameras and other known apparatus mitigating shadows and other artifacts. Processes, systems and methods likewise are disclosed.

### CROSS-REFERENCE TO RELATED APPLICATION

This applications claims full Paris Convention and related priority rights from U.S. Provisional Ser. No. 60//825,893 filed Sep. 15, 2006, which was entitled HIGHER DEFINITION ENDOSCOPE, by the present inventor, and said document is expressly incorporated herein by reference as if fully set forth herein. This present disclosure relates to methods for using medical devices, and apparatus for use clinically with patients, namely endoscopic devices. More specifically, the instant teachings are directed to a set of methods, systems and devices for illumination of the viewing area during use of endoscopic devices and related procedures.

### FIELD OF THE DISCLOSURE

### Background Art

Endoscopic devices are well-known in the art and perform essential functions in conjunction with therapies addressing numerous disease states. Likewise, endoscopic devices are used in many industries wherein minimally invasive inspection is imperative, for example, such devices are used in medical health care which also benefits from the minimal invasiveness of these devices.

The endoscope provides a guidable flexible viewing tip which provides image data output for viewing and/or processing. The area of inspection or investigation that is within the range of the viewing tip often requires illumination and many endoscopes provide illumination at the tip via fiber optics lines and the like. Too often the image data output appears flat, and topographic details are hard to discern, making usage of the same by medical professionals overly challenging and inconsistent in terms of results.

It remains a longstanding need and concomitant desideratum to have an endoscopic device which transmits image data output with better topographic details.

### SUMMARY OF THE DISCLOSURE

Briefly stated novel enhanced high definition endoscopy is facilitated by multiple illumination sources and a plurality of illumination sources and a plurality of illumination zones, whereby improved images are generated and image date output is communicated to eye pieces, computer screens, cameras and other known apparatus mitigating shadows and other artifacts. Processes, systems and methods likewise are disclosed.

According to embodiments, the present disclosure provides an endoscopic device with controlled area specific illumination.

According to embodiments, the present disclosure provides an endoscopic device with controlled area specific illumination in selected ranges of wavelengths

According to embodiments, the present disclosure provides an endoscopic device with controlled area specific illumination in selected illumination intensities.

According to embodiments, the present disclosure provides an endoscopic illumination method of separated illumination whereby topographic details are enhanced.

According to embodiments, the present disclosure provides an endoscopic illumination system of controlled separated illumination.

According to embodiments, the image data output is at least optical

According to embodiments, the image data output is at least digital.

In some exemplary implementations at least one of the illuminations is laser output.

According to embodiments, at least one of the illuminations is LED output.

According to embodiments, at least one of the illuminations is variable intensity.

According to embodiments, at least one of the illuminations is variable; and The image data output is provided to at least one of an eye piece, a computer screen, a camera, a digital camera, a video camera, a CCD camera, a CMOS camera, an optical sensor, and a computer.

According to embodiments, there is provided an improved high definition endoscopic device, comprising, in combination: a body extended into a truncated and guidable neck portion; a tip located of a distal end of the guidable neck having an imaging lens and at least two illumination zones; and at least an illumination source, with controlled area specific illumination, in selected ranges of wavelengths and in selected illumination intensities, whereby topographic details are enhanced of an image produced.

According to embodiments, there is provided an endoscopic device with controlled area specific illumination in selected illumination intensities, whereby using separated illumination topographic details are enhanced, comprising, in combination:

According to embodiments, there is provided a process for performing endoscopic procedures, which comprises, in combination; providing an enhanced endoscopic device with controlled area specific illumination, in selected ranges of wavelengths and in selected illumination intensities, wherein generated image data output is at least one of optical, digital, laser-based and LED output.

According to embodiments, other features and advantages of the present invention will be set forth, in part, in the descriptions which follow and the accompanying drawings, wherein preferred embodiments and some exemplary implementations of the present invention are described and shown, and in part, will become apparent to those skilled in the art upon examination of the following detailed description taken in conjunction with the accompanying drawings or may be learned by practice of the present invention. The advantages of the present invention may be realized and attained by means of the instrumentalities and combinations of elements and instrumentalities particularly pointed out in the appended claims.

### BRIEF DESCRIPTION OF THE DRAWINGS

FIG. 1 is side view diagram of an endoscopic device, according to embodiments of the present disclosure;

FIG. 2 is another side view diagram of an endoscopic device, according to embodiments of the present disclosure;

FIG. 3 is a cut-away view of section “A” of the endoscopic device shown in FIG. 1, according to embodiments of the present disclosure;

FIG. 4 is a front view of the tip of an endoscopic device, according to embodiments of the present disclosure;

FIG. 5 is a front view of another tip of an endoscopic device, according to embodiments of the present disclosure;

FIG. 6 is a front view of another tip of an endoscopic device, according to embodiments of the present disclosure;

FIG. 7 is a diagram of system components of an endoscopic device, according to embodiments of the present disclosure;

FIG. 8 is a flow chart of a sequence of operation of an aspect of illumination of an endoscopic device, according to embodiments of the present disclosure;

FIG. 9 is a flow chart of a sequence of operation of an aspect of illumination of an endoscopic device, according to embodiments of the present disclosure;

FIG. 10 is a flow chart of a sequence of operation of an aspect of illumination of an endoscopic device, according to embodiments of the present disclosure;

FIG. 11 is a flow chart of a sequence of operation of an aspect of illumination of an endoscopic device, according to embodiments of the present disclosure;

FIG. 12 is a flow chart of a sequence of operation of an aspect of illumination of an endoscopic device, according to embodiments of the present disclosure; and

It should be appreciated that for simplicity and clarity of illustration, elements shown in the Figures have not necessarily been drawn to scale. For example, the dimensions of some of the elements are exaggerated relative to each other for clarity. Further, where considered appropriate, reference numerals have been repeated among are merely illustrative, and without limiting the instant teachings.

### DESCRIPTION OF PREFERRED IMPLEMENTATIONS OF THE DISCLOSURE

Detailed embodiments are disclosed herein, however, it is to be understood that the disclosed embodiments are merely exemplary implementations of the invention, which may be embodied in various forms. Therefore, specific structural and functional details disclosed herein are not to be interpreted as limiting, but merely as a basis for claims and as a representative basis for teaching one skilled in the art to variously employ the present invention in virtually any appropriately detailed structure.

The present inventor has addressed issues relating to the paucity of useful imagery available with extant endoscopy systems. According to the instant teachings, the present inventor uses enhanced illumination, among other things, to mitigate shadows which often degrade the quality and accuracy of imagery generated by current systems.

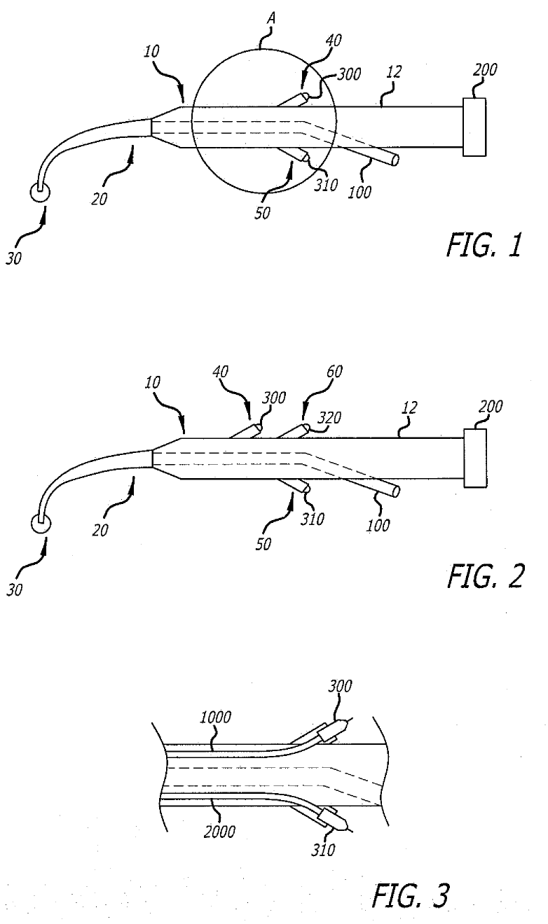

Turning now to FIG. 1, endoscopic devices according to the teachings of the current disclosure are shown generally at 10. Body 12 provides support for guidable neck 20 at the end of the guidable neck is tip 30. Referring now also to FIGS. 2 and 3, there is shown first illumination port 40 wherein a first illumination source 300 is optically connected to light guide 100. A second illumination port 50 is connected to light guide 200 and receives illumination from a second illumination source 310.

Illumination sources include, but are not limited to, incoherent light, coherent light, quasi-coherent, visible spectrum light, electromagnetic radiation “EM” above or below the visual threshold, EM radiation at a selected range of wavelengths, EM radiation of constant or variable intensity. Light of variable or fixed nits are likewise within the scope of embodiments of the instant disclosure.

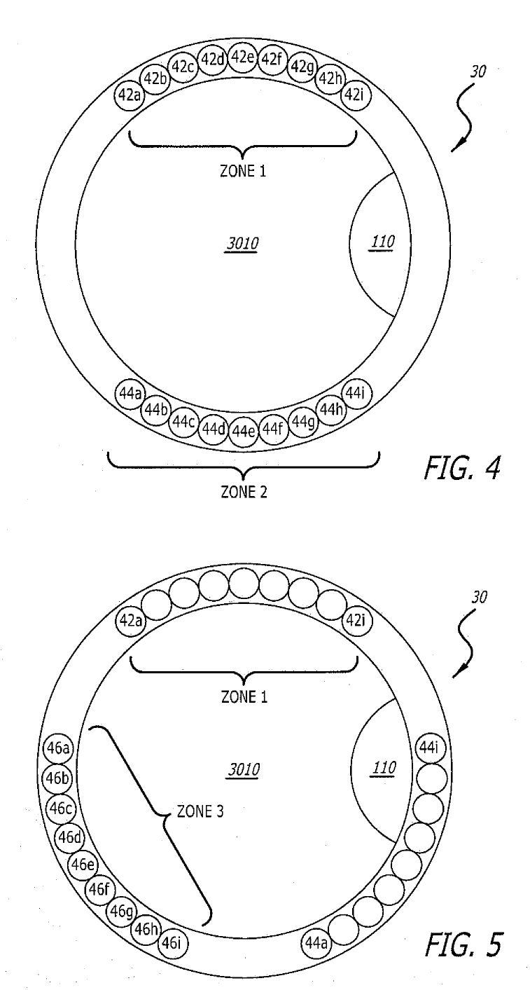

Referring now to FIG. 4, at least two illumination zones are shown. Within the tip is an imaging lens 3010 and illumination zones, shown in FIG. 4 as zones “1” and “2”. Illumination is provided to flexible light guides such as fiber optic cables or photonic material. Those light guides receive the output of an illumination source. A working channel 110 is provided through which irrigation, aspiration, radiation, cautery, steering, cutting and grasping may occur.

A viewing channel is provided within the body 12 (not shown) connected to a view end 200. At the viewing end an eye, camera, video camera, digital camera and/or sensor may be connected. Images may be projected or displayed on a CCTV, monitor, display or computer screen. A splitter, which is known in the art, may be used to provide an optical data stream to multiple receiving or processing devices. Processing devices include the human brain, computers and sensors.

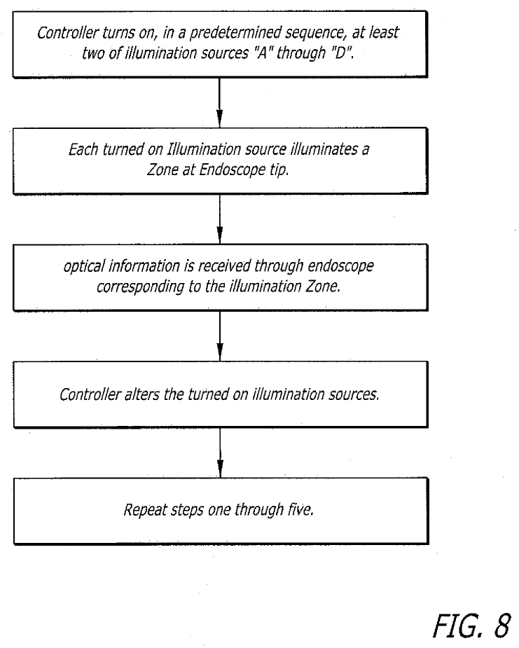

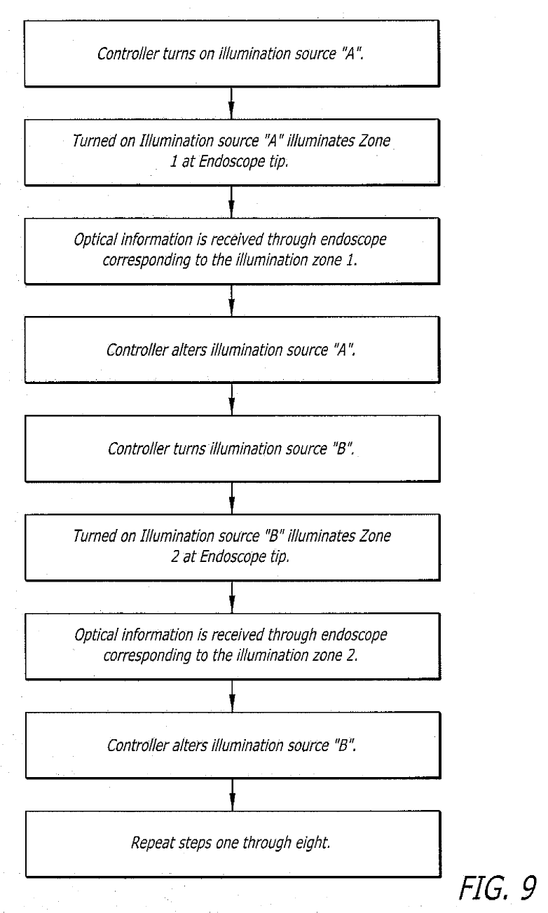

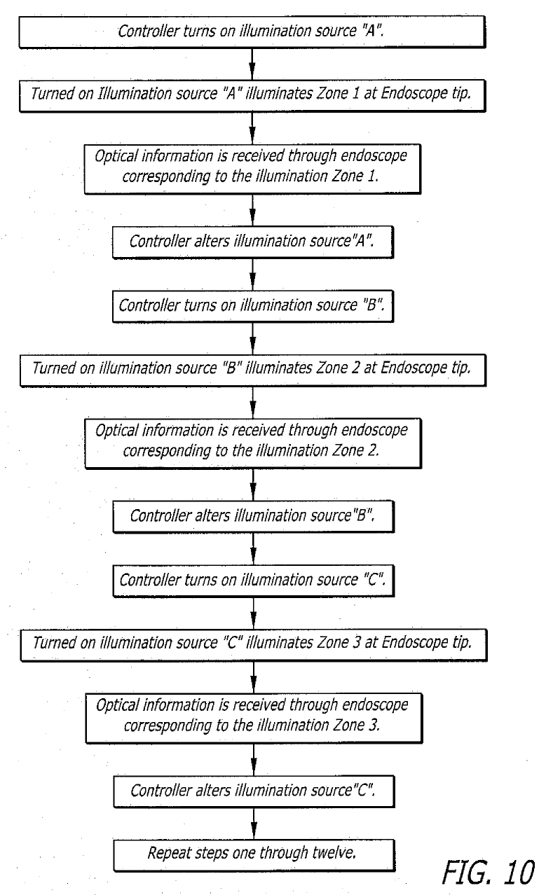

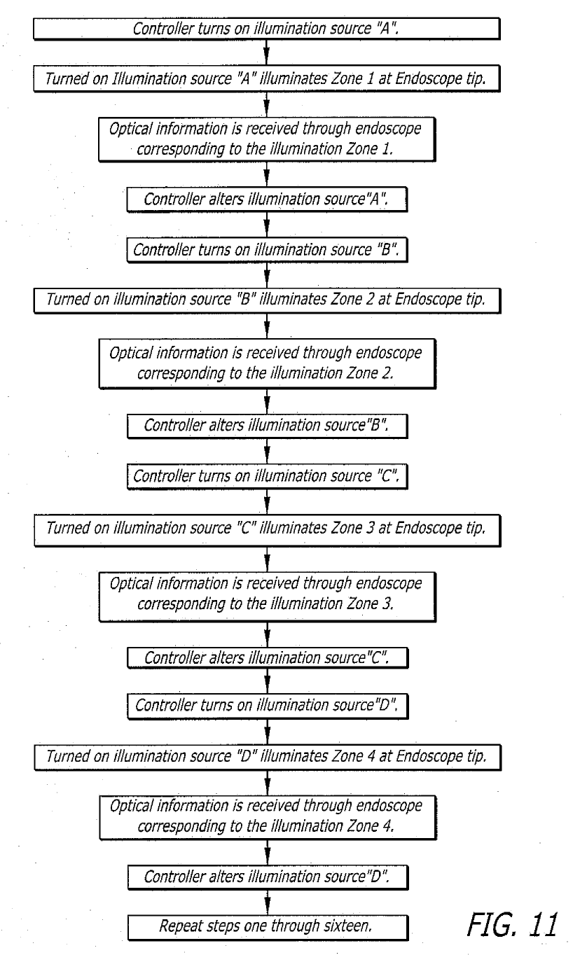

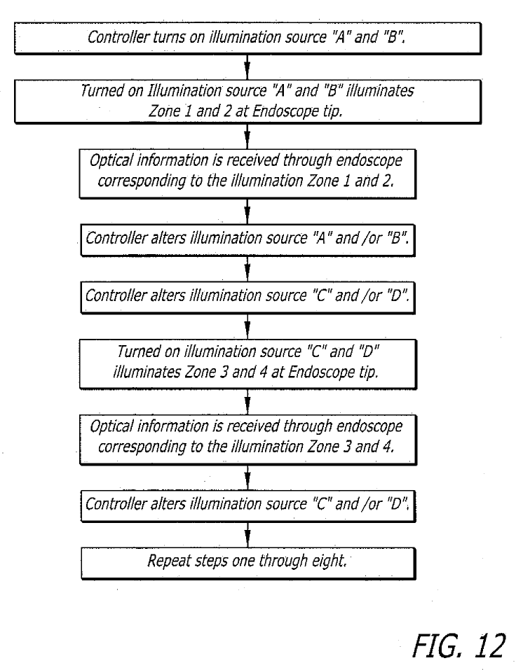

The turning on or off of the first or the second illumination source may be done manually, by hand or foot control, or automatically via a programmed switch or a computer as detailed in the flow charts in FIGS. 8 through 12.

FIGS. 4-6 show a tip 30 of endoscope implementations. A lens 3010 is provided to receive the optical image data from an area being illuminated by the device. By having distinct illumination in at least two zones features such as topography, geometry, heat signature, absorption to various EM radiation types can be ascertained.

An illumination zone consists of an area fed by light guides which terminate in a distinct area around the tip 30. For example, zone 1 is fed EM radiation by a light guide 1000 which is shown as a group of apertures 42a-42i forming zone 1. Zone 2 is fed EM radiation by a light guide 2000 which are shown as a group of apertures 44a-44i forming zone 2. The open end 110 of the working channel 100 is provided at the tip 30. The type, size or members forming the light guide are variable and may be selected form available light guides which transmit the type of illumination needed for a particular deployment, as understood by those skilled in the art.

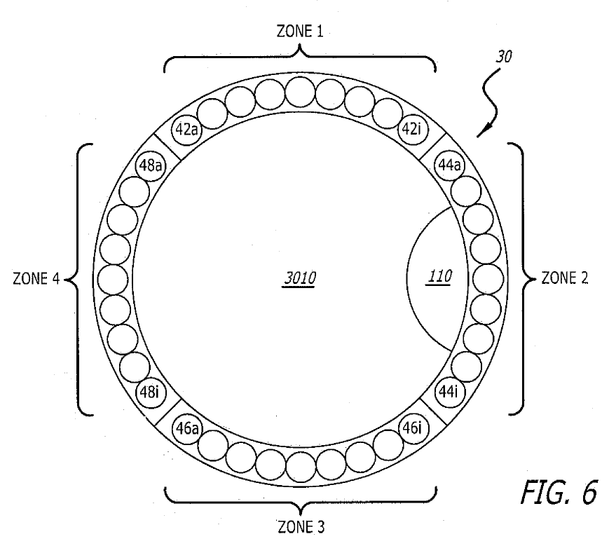

In FIG. 5 illumination zone 3 is added. Zone 3 is fed EM radiation by a light guide 3000 which is also shown as a group apertures 46a-46i forming zone 3. In FIG. 6 illumination zone 4 is added. Zone 4 is fed EM radiation by a light guide 4000 which is also shown as a group apertures 48a-48i forming zone 4.

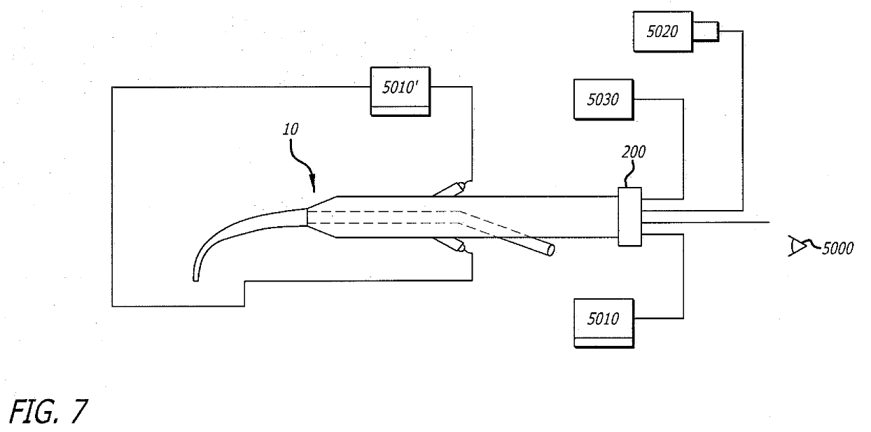

Shown in FIG. 7 are some components of an implementation of the illumination system disclosed herein. An endoscope 10 with a view end 200 the view end supplying optical data to on or more of an eye 5000, a computer 5010, a camera or video camera 5020, a sensor 5030. Such optical data can be further processed an can be stored analog and/or digital. Further, a computer 5010′ or switch which acts as a controller can be used to automatically sequence, adjust or alter the illumination sources. The computer used to receive or display optical data may also be the computer used to control the illumination source.

FIGS. 8-12 provide flow charts of some aspects of automated control of the illumination, the steps of which are hereby incorporated by this reference.

Since certain changes may be made in the above apparatus without departing from the scope of the invention herein involved, it is intended that all matter contained in the above description, as shown in the accompanying drawings, shall be interpreted in an illustrative, and not a limiting sense.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. An improved high definition endoscopic device, comprising, in combination: a body extended into a truncated and guidable neck portion; a tip located of a distal end of the guidable neck having an imaging lens and at least two illumination zones; and at least an illumination source, with controlled area specific illumination, in selected ranges of wavelengths and in selected illumination intensities, whereby topographic details are enhanced of an image produced.

2. The improved high definition endoscope or claim 1, having a plurality of illumination sources.

3. The improved high definition endoscope of claim 2, having a plurality of illumination zones.

4. The improved high definition endoscope of claim 3, illumination sources further comprising at least one of, incoherent light, coherent light, quasi-coherent, visible spectrum light, electromagnetic radiation “EM” above or below the visual threshold, EM radiation at a selected range of wavelengths, EM radiation of constant or variable intensity, and light of variable or fixed nits.

5. The improved high definition endoscope of claim 4, comprised of at least three illumination zones.

6. The improved high definition endoscope of claim 5, comprised of at least four illumination zones.

7. In a novel enhanced endoscopic device with controlled area specific illumination in selected illumination intensities, whereby using separated illumination topographic details are improved, the improvement comprising using multiple illumination zones.

8. The device of claim 7, having two illumination zones.

9. The device of claim 8, having three illumination zones.

10. The device of claim 9, having at least four illumination zones.

11. A process for performing endoscopic procedures, which comprises, in combination:
providing an enhanced endoscopic device with controlled area specific illumination, in selected ranges of wavelengths and in selected illumination intensities,
wherein generated image data output is at least one of optical, digital, laser-based and LED output.

12. The process of claim 11, the generating step further comprising:
A Controller turns on, in a predetermined sequence, at least two of illumination sources “A” through “D”; each turned on illumination source illuminates a zone at Endoscope tip; optical information is receiving through endoscope corresponding to the illumination zone; controller alters the turned on illumination sources; and repeating the steps.

13. The process of claim 11, the generating step alternately comprising:
A Controller turns on illumination source “A”; turned on illumination source “A” illuminates zone 1 at endoscope tip; optical information is received through endoscope corresponding to the illumination zone 1; controller alters illumination source “A”; controller turns illumination source “B”; turned on illumination source “B” illuminates zone 2 at endoscope tip; optical information is received through endoscope corresponding to the illumination zone 2; controller alters illumination source “B”; and repeating the steps.

14. The process of claim 11, the generating step alternately comprising:
A Controller turns on illumination source “A”; turned on illumination source “A” illuminates zone 1 at endoscope tip; optical information is received through endoscope corresponding to the illumination zone 1; controller alters illumination source “A”; controller turns illumination source “B”; turned on illumination source “B” illuminates zone 2 at endoscope tip; optical information is received through endoscope corresponding to the illumination zone 2; controller alters illumination source “B”; controller alters illumination source “D”; and repeating the steps.

15. The process of claim 11, the generating step alternately comprising:
A Controller turns on illumination source “A” and “B”; turned on illumination source “A” and “B” illuminates Zone 1 and 2 at endoscope tip; optical information is received through endoscope corresponding to the illumination zone 1 and 2; controller alters illumination source “A” and/or “B”; controller alters illumination source “C” and/or “D”; turned on illumination source “C” and “D” illuminates zone 3 and 4 at endoscope tip; optical information is received through endoscope corresponding to the illumination source “C” and/or “D” and repeating the steps.

16. The process of claim 15, wherein at least one of the illuminations is of variable intensity.

17. The process of claim of claim 16, wherein image data output is provided to at least one of an eye piece, a computer screen, a camera, a digital camera, a video camera, a CCD camera, a CMOS camera, an optical sensor, and a computer.

18. A product by the process of claim 17.

19. A product by the process of claim 13.

20. A product by the process of claim 14.
